(19) United States
(12) Patent Application Publication
(10) Pub. No.: US 2025/0121128 A1
(43) Pub. Date: Apr. 17, 2025

(54) ANTIMICROBIAL INTRAVENOUS SET

(71) Applicant: CareFusion 303, Inc., San Diego, CA (US)

(72) Inventor: Gene E. Mason, La Habra Heights, CA (US)

(21) Appl. No.: 18/488,392

(22) Filed: Oct. 17, 2023

(51) Int. Cl. A61M 5/142; A61L 2/08; A61L 2/10; A61L 2/26; A61M 39/16; G02B 1/04; G02B 6/02

(52) U.S. Cl. CPC A61M 5/142; A61L 2/084; A61L 2/10; A61L 2/26; A61M 39/16; G02B 1/046; G02B 6/02033; A61L 2202/11; A61M 2039/167; A61M 2209/10

(57) ABSTRACT

An intravenous administration set is disclosed. The intravenous administration set includes a fluid container that contains a medicament and has an outlet on a proximal end thereof, a patient access tip that has an inlet on a distal end and a proximal end configured to be inserted into a patient, a tubing that has a distal end coupled to the outlet of the fluid container and a proximal end coupled to the distal end of the patient access tip, a pump coupled to the tubing between the fluid container and the patient access tip, and a light source emitting a light along the tubing toward the patient access tip.

### TECHNICAL FIELD

The present disclosure generally relates to an intravenous pump set and, in some embodiments, to an antimicrobial intravenous pump set.

### SUMMARY

The disclosed subject matter relates to an antimicrobial intravenous set. In certain embodiments, the intravenous administration set includes a fluid container containing a medicament and having an outlet on a proximal end thereof; a patient access tip having an inlet on a distal end and a proximal end configured to be inserted into a patient; a tubing having a distal end coupled to the outlet of the fluid container and a proximal end coupled to the distal end of the patient access tip; a pump coupled to the tubing between the fluid container and the patient access tip; and a light source emitting a light along the tubing toward the patient access tip.

In certain embodiments, the light is emitted through the tubing. In certain embodiments, the light is transmitted via a fiber optic filament. In certain embodiments, the fiber optic filament comprises polystyrene. In certain embodiments, the fiber optic filament extends from the pump to the patient access tip. In certain embodiments, the fiber optic filament extends partially from the pump to the patient access tip. In certain embodiments, the light is emitted via an optical connection. In certain embodiments, the light is transmitted via tubing lumens. In certain embodiments, the light is transmitted through a tubing wall. In certain embodiments, the light source is disposed in the pump. In certain embodiments, the light travels in a proximal direction from the pump toward the patient access tip.

In certain embodiments, the light has antimicrobial properties. In certain embodiments, the light is an indigo light. In certain embodiments, the indigo light has a wavelength from the visible light spectrum of approximately 405 nm. In certain embodiments, the light source constantly emits the light. In certain embodiments, the fluid container and the patient access tip are in fluid communication through the tubing. In certain embodiments, the pump is coupled to the tubing closer to the fluid container than the patient access tip.

In certain embodiments, the intravenous administration set includes a fluid container containing a medicament and having an outlet on a proximal end thereof; a patient access tip having an inlet on a distal end and a proximal end configured to be inserted into a patient; a tubing having a distal end coupled to the outlet of the fluid container and a proximal end coupled to the distal end of the patient access tip; a pump coupled to the tubing between the fluid container and the patient access tip; and a light source emitting a light along the tubing to the patient access tip, wherein the light is emitted through the tubing, and wherein the light has antimicrobial properties.

In certain embodiments, the light is transmitted via a fiber optic filament. In certain embodiments, the light is an indigo light.

### BRIEF DESCRIPTION OF THE DRAWINGS

The foregoing summary, as well as the following detailed description of embodiments of the antimicrobial intravenous set, will be better understood when read in conjunction with the appended drawings of an exemplary embodiment. It should be understood, however, that the invention is not limited to the precise arrangements and instrumentalities shown.

In the drawings:

FIG. 1 depicts a patient receiving an infusion of a medical fluid using an IV pump;

FIG. 2 is a side view of an IV set in accordance with exemplary embodiments of the present disclosure;

FIG. 3 is a side view of an IV pump with an optical window in accordance with exemplary embodiments of the present disclosure; and

FIG. 4 is a top view of an IV set and patient access tip in accordance with exemplary embodiments of the present disclosure.

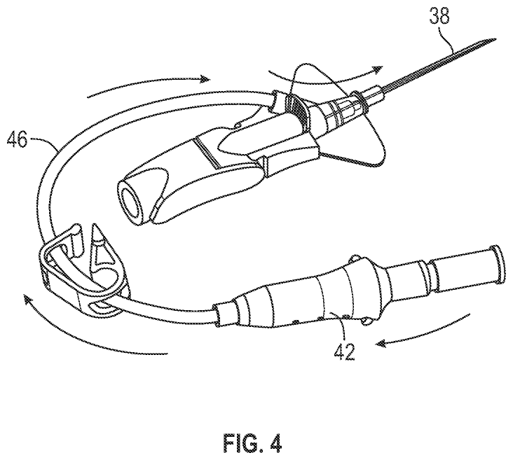

### DETAILED DESCRIPTION

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding. Reference numbers may have letter suffixes appended to indicate separate instances of a common element while being referred to generically by the same number without a suffix letter.

While the following description is directed to administration of medical fluid by utilizing the disclosed peristaltic pumps, it is to be understood that this description is only an example of usage and does not limit the scope of the claims. Various aspects of the disclosed peristaltic pumps may be used in any application where it is desirable to administer the flow of fluid.

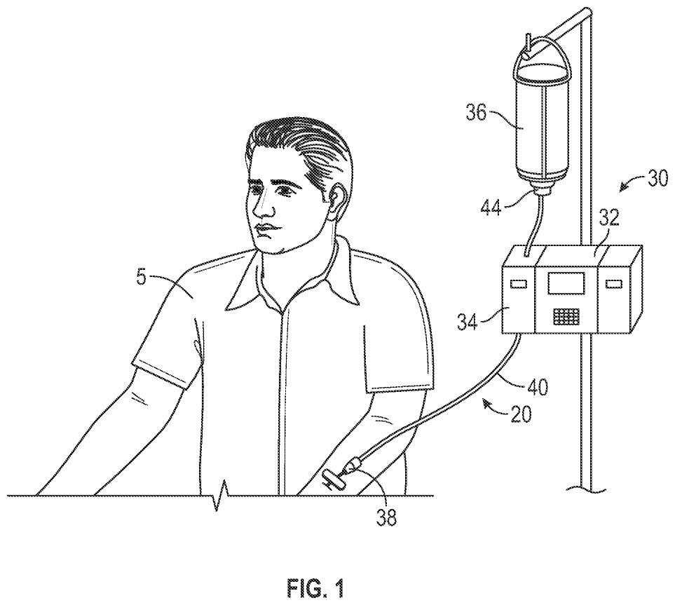

FIG. 1 depicts a patient 5 receiving an infusion of a medical fluid using an IV pump 30. In the depicted example, the IV pump 30 is delivering a medical fluid from a fluid container 36 to the patient 5. A fluid container 36 is hung at or above the patient's head and connected via an IV set 20 to the IV pump module 34 and then to the patient 5. In some embodiments, the IV pump 30 includes a control unit 32 and a pumping module 34. Suitable IV pump configurations and systems are described, for example, in U.S. patent application Ser. No. 17/586,619, the entire disclosure of which is hereby incorporated by reference.

The pumping module 34 can include a peristaltic pump to administer the medical fluid from the fluid container 36 to the patient 5. During operation of the peristaltic pump, it may be desirable to monitor the volume pumped by the peristaltic pump. In some applications, the peristaltic pump can include antimicrobial functionalities, as described in more detail below.

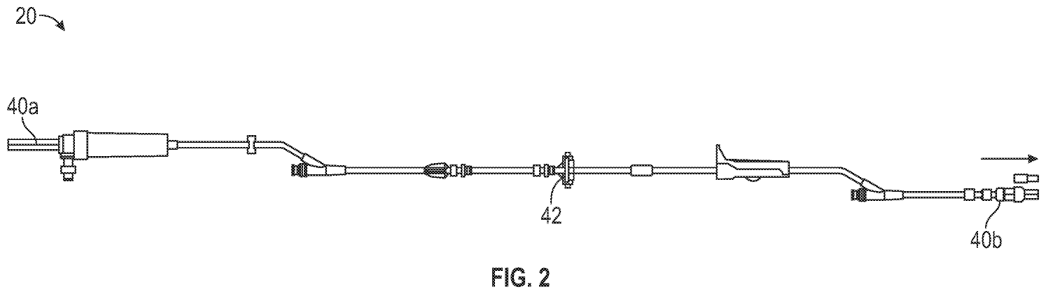

As shown in FIG. 1, the IV set 20 includes a fluid container 36, a patient access tip 38, a tubing 40, the IV pump 30 and a light source 42 (FIG. 2). It is noted that, in the context of this disclosure, the terms “distal” and “proximal” are used in reference to a position relative to the patient 5. The fluid container 36 may contain a medical fluid (e.g., a saline solution or a liquid medication) for intravenous administration. The fluid container 36 may be rigid, as shown in FIG. 1, or may alternatively be a flexible bag containing the medical fluid. The fluid container 36 includes an outlet 44 on a proximal end thereof. The outlet 44 may be a generally circular member extending through the proximal end of the fluid container 36. The outlet 44 may have a septum (not shown) extending across to prevent the medical fluid from passing therethrough. The outlet 44 may have an inner or outer surface configured to allow other components to be coupled thereto, as described below in more detail.

The tubing 40 may be a plastic or rubber capable of carrying the medical fluid therethrough. The tubing 40 may have a rigidity sufficient to maintain a generally cylindrical shape when an exterior force is not acting on it. However, the tubing 40 may be flexible enough to bend and change a cross-section thereof in response to a force acting thereon by, for example, a pump as described in more detail below.

As shown in FIG. 2, the tubing 40 may have a distal end 40a and a proximal end 40b. The distal end of the tubing 40 may be configured to be coupled to the outlet 44. The distal end of the tubing 40 may include threading or protrusions which are received by corresponding threading or depressions of the outlet 44. As such, the outlet 44 and the tubing 40 may be coupled by threaded, snap-fit, adhesive, weld, or any other suitable form of coupling for the specific application.

The proximal end of the tubing 40 may be coupled to a distal end of a patient access tip 38. The patient access tip 38 may have an inlet on the distal end thereof. The distal end of the access tip 38 may include threading or protrusions which are received by corresponding threading or depressions of the tubing 40. As such, the outlet 44 and the tubing 40 may be coupled by threaded, snap-fit, adhesive, weld, or any other suitable form of coupling for the specific application.

The patient access tip 38 have a proximal end configured to be inserted into the patient 5 during administration of the medical fluid. The proximal end may be a needle sized and shaped to deliver the medical fluid at the desired rate. The proximal end may be a 17 gauge needle, a 18 gauge needle, a 19 gauge needle, a 20 gauge needle, a 21 gauge needle, a 22 gauge needle, a 23 gauge needle, a 24 gauge needle, a 25 gauge needle, a 26 gauge needle, a 27 gauge needle, a 28 gauge needle, a 29 gauge needle, or a 30 gauge needle. In some embodiments, the fluid container 36 and the patient access tip 38 are in fluid communication through the tubing 40.

The IV pump 30 may be coupled to the tubing 40 between the fluid container 36 and the patient access tip 38. As shown in FIG. 1, the IV pump 30 may be coupled to the tubing 40 closer to the fluid container 36 than the patient access tip 38. The IV pump 30 may include internal components that engage the tubing 40 to manipulate the cross-section such that flow of the medical fluid can be modulated and controlled according to the specifical application of the IV set 20.

As shown in FIG. 2, a light source 42 may emit a light along the tubing 40 toward the patient access tip 38. In some embodiments, the light has antimicrobial properties. For example, the light may kill pathogens. This may reduce bacterial colonization at a patient access site during administration of the medical fluid. The light may be an indigo light. The indigo light may have a wavelength from the visible light spectrum of at least 405 nm. In some embodiments, the indigo light has a wavelength from the visible light spectrum of approximately 405 nm. 415 nm, 425 nm, 435 nm, 445 nm, or 450 nm. In some embodiments, the indigo light has a wavelength from the visible light spectrum of between 405 nm and 425 nm, 410 nm and 420 nm, 405 nm and 450 nm, 415 nm and 440 nm, and 425 nm and 430 nm. In some embodiments, the light source 42 constantly emits the light. In some embodiments, the light source 42 intermittently emits the light.

Application of the light may reduce bacterial colonization without the need to introduce potentially harsh chemicals or other antimicrobial treatments and may avoid using expensive silver or platinum-based materials with natural sanitizing properties. This may increase catheter dwell times, thereby allowing the IV set 20 to administer more of the medical fluid per use and reducing the number of insertions. Further, the indigo light is not harmful to human eyes like other commonly used lights in this space (e.g., UV-based systems). Description of the use of indigo light proving effective against antimicrobial resistant organisms can be found in at least U.S. Pat. No. 9,039,966, the entirety of which is hereby incorporated by reference.

The light may be emitted through the tubing 40 by the light source 42. In some embodiments, the light is transmitted via a fiber optic filament 46. The fiber optic filament 46 may be disposed within the tubing 40, fixed to an interior wall of the tubing 40, embedded in a wall of the tubing 40, or fixed to an exterior wall of the tubing 40, or any combination thereof. The fiber optic filament 46 may comprise a suitable material for transmitting the light emitted from the light source 42, for example polystyrene. The fiber optic filament 46 may extend from the IV pump 30 to the patient access tip 38. In some embodiments, the fiber optic filament 46 extends partially from the IV pump 30 to the patient access tip 38. The fiber optic filament 46 can terminate at the most clinically advantageous depth wherever infections most likely occur or wherever maximum catheter flexibility is balanced with antimicrobial effectiveness.

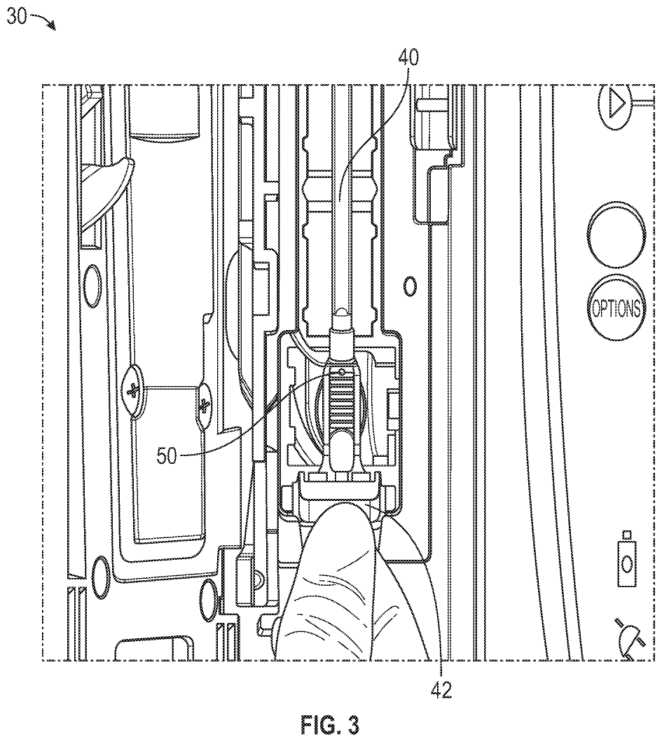

As shown in FIG. 3, the light source 42 may be disposed in the IV pump 30. In some embodiments, the light may be transmitted via an optical connection 48. The optical connection 48 may be disposed between the fluid container 36 and the patient access tip 38 and may transmit the light toward the patient access tip. The optical connection 48 may include a body with an import 50 and an export 52. The import 50 may receive a proximal end of a first segment of the tubing 40 and the export 52 may receive a distal end of a second segment of the tubing 40 to allow the medical fluid to flow therethrough. The optical connection may transmit the light through the second portion of the tubing 40 in any manner discussed above in connection with the fiber optic filament 46 (e.g., inside the inner wall or inside the wall of the tubing 40). In some embodiments, the light may be transmitted via an optical lumen (not shown) extending along the tubing 40. In some embodiments, the light may be transmitted via the wall of the tubing 40 (not shown).

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 18. The other clauses can be presented in a similar manner.

Clause 1: An intravenous administration set comprising: a fluid container containing a medicament and having an outlet on a proximal end thereof; a patient access tip having an inlet on a distal end and a proximal end configured to be inserted into a patient; a tubing having a distal end coupled to the outlet of the fluid container and a proximal end coupled to the distal end of the patient access tip; a pump coupled to the tubing between the fluid container and the patient access tip; and a light source emitting a light along the tubing toward the patient access tip.

Clause 2: The intravenous administration set of clause 1, wherein the light is emitted through the tubing.

Clause 3: The intravenous administration set of clause 2, wherein the light is transmitted via a fiber optic filament.

Clause 4: The intravenous administration set of clause 3, wherein the fiber optic filament comprises polystyrene.

Clause 5: The intravenous administration set of clause 3, wherein the fiber optic filament extends from the pump to the patient access tip.

Clause 6: The intravenous administration set of clause 3, wherein the fiber optic filament extends partially from the pump to the patient access tip.

Clause 7: The intravenous administration set of clause 2, wherein the light is emitted via an optical connection.

Clause 8: The intravenous administration set of clause 2, wherein the light is transmitted via tubing lumens.

Clause 9: The intravenous administration set of clause 2, wherein the light is transmitted through a tubing wall.

Clause 10: The intravenous administration set of clause 1, wherein the light source is disposed in the pump.

Clause 11: The intravenous administration set of clause 10, wherein the light travels in a proximal direction from the pump toward the patient access tip.

Clause 12: The intravenous administration set of clause 1, wherein the light has antimicrobial properties.

Clause 13: The intravenous administration set of clause 12, wherein the light is an indigo light.

Clause 14: The intravenous administration set of clause 13, wherein the indigo light has a wavelength from the visible light spectrum of approximately 405 nm.

Clause 15: The intravenous administration set of clause 1, wherein the light source constantly emits the light.

Clause 16: The intravenous administration set of clause 1, wherein the fluid container and the patient access tip are in fluid communication through the tubing.

Clause 17: The intravenous administration set of clause 1, wherein the pump is coupled to the tubing closer to the fluid container than the patient access tip.

Clause 18: An intravenous administration set comprising: a fluid container containing a medicament and having an outlet on a proximal end thereof; a patient access tip having an inlet on a distal end and a proximal end configured to be inserted into a patient; a tubing having a distal end coupled to the outlet of the fluid container and a proximal end coupled to the distal end of the patient access tip; a pump coupled to the tubing between the fluid container and the patient access tip; and a light source emitting a light along the tubing to the patient access tip, wherein the light is emitted through the tubing, and wherein the light has antimicrobial properties.

Clause 19: The intravenous administration set of clause 18, wherein the light is transmitted via a fiber optic filament.

Clause 20: The intravenous administration set of clause 18, wherein the light is an indigo light.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous administration set comprising:
a fluid container containing a medicament and having an outlet on a proximal end thereof;
a patient access tip having an inlet on a distal end and a proximal end configured to be inserted into a patient;
a tubing having a distal end coupled to the outlet of the fluid container and a proximal end coupled to the distal end of the patient access tip;
a pump coupled to the tubing between the fluid container and the patient access tip; and
a light source emitting a light along the tubing toward the patient access tip.

2. The intravenous administration set of claim 1, wherein the light is emitted through the tubing.

3. The intravenous administration set of claim 2, wherein the light is transmitted via a fiber optic filament.

4. The intravenous administration set of claim 3, wherein the fiber optic filament comprises polystyrene.

5. The intravenous administration set of claim 3, wherein the fiber optic filament extends from the pump to the patient access tip.

6. The intravenous administration set of claim 3, wherein the fiber optic filament extends partially from the pump to the patient access tip.

7. The intravenous administration set of claim 2, wherein the light is emitted via an optical connection.

8. The intravenous administration set of claim 2, wherein the light is transmitted via tubing lumens.

9. The intravenous administration set of claim 2, wherein the light is transmitted through a tubing wall.

10. The intravenous administration set of claim 1, wherein the light source is disposed in the pump.

11. The intravenous administration set of claim 10, wherein the light travels in a proximal direction from the pump toward the patient access tip.

12. The intravenous administration set of claim 1, wherein the light has antimicrobial properties.

13. The intravenous administration set of claim 12, wherein the light is an indigo light.

14. The intravenous administration set of claim 13, wherein the indigo light has a wavelength from a visible light spectrum of approximately 405 nm.

15. The intravenous administration set of claim 1, wherein the light source constantly emits the light.

16. The intravenous administration set of claim 1, wherein the fluid container and the patient access tip are in fluid communication through the tubing.

17. The intravenous administration set of claim 1, wherein the pump is coupled to the tubing closer to the fluid container than the patient access tip.

18. An intravenous administration set comprising:
a fluid container containing a medicament and having an outlet on a proximal end thereof;
a patient access tip having an inlet on a distal end and a proximal end configured to be inserted into a patient;
a tubing having a distal end coupled to the outlet of the fluid container and a proximal end coupled to the distal end of the patient access tip;
a pump coupled to the tubing between the fluid container and the patient access tip; and
a light source emitting a light along the tubing to the patient access tip,
wherein the light is emitted through the tubing, and
wherein the light has antimicrobial properties.

19. The intravenous administration set of claim 18, wherein the light is transmitted via a fiber optic filament.

20. The intravenous administration set of claim 18, wherein the light is an indigo light.
